(19) United States
(12) Patent Application Publication
(10) Pub. No.: US 2007/0083218 A1
(43) Pub. Date: Apr. 12, 2007

(54) Coated ligating clip

(76) Inventor: Steven A. Morris, Cary, NC (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(21) Appl. No.: 11/247,164

(22) Filed: Oct. 12, 2005

(51) Int. Cl. A61B 17/08

(52) U.S. Cl. 606/157

(57) ABSTRACT

A polymeric, surgical clip having first and second curved leg members joined at their proximal end by a hinge portion and movable from an open position to a closed position for clamping a vessel between curved opposing inner surfaces. The clip includes a coating on its exterior which reduces the friction between the leg members and complementary locking mechanisms disposed at the distal end portions of the leg members, and stabilizes closing of the clip. The coating can be any coating, including a polymer coating such as one which includes polytetrafluoroethylene. The coating may be applied using a solvent such as HFC43-10.

### FIELD OF THE INVENTION

The present invention relates generally to medical and surgical devices. More particularly, the present invention relates to polymer clips used for ligation of tissue, body vessels or members, and other surgical applications.

### BACKGROUND OF THE INVENTION

Many surgical procedures require vessels or other tissues of the human body to be ligated during the surgical process. For example, many surgical procedures require cutting blood vessels (e.g., veins or arteries), and these blood vessels may require ligation to reduce and/or stop bleeding. In some instances, a surgeon may wish to ligate the vessel temporarily to reduce blood flow to the surgical site during the surgical procedure. In other instances a surgeon may wish to permanently ligate a vessel. Ligation of vessels or other tissues can be performed by closing the vessel with a ligating clip, or by suturing the vessel with surgical thread. The use of surgical thread for ligation requires complex manipulations of the needle and suture material to form the knots required to secure the vessel. Such complex manipulations are time-consuming and difficult to perform, particularly in endoscopic surgical procedures, which are characterized by limited space and visibility. By contrast, ligating clips are relatively easy and quick to apply. Accordingly, the use of ligating clips in endoscopic as well as open surgical procedures has grown dramatically.

Various types of hemostatic and aneurysm clips are used in surgery for ligating blood vessels or other tissues to stop the flow of blood. Such clips have also been used for interrupting or occluding ducts and vessels in particular surgeries such as sterilization procedures. Typically, a clip is applied to the vessel or other tissue by using a dedicated mechanical instrument commonly referred to as a surgical clip applier, ligating clip applier, or hemostatic clip applier. Generally, the clip is left in place after application to the tissue until hemostasis or occlusion occurs. At some point thereafter, the clip is removed by using a separate instrument dedicated for that purpose, i.e., a clip removal instrument.

Ligating clips can be classified according to their geometric configuration (e.g., symmetric clips or asymmetric clips), and according to the material from which they are manufactured (e.g., metal clips or polymeric clips). Symmetric clips are generally “U” or “V” shaped and thus are substantially symmetrical about a central, longitudinal axis extending between the legs of the clip. Symmetric clips can be constructed from metals such as stainless steel, titanium, tantalum, or alloys thereof. By means of a dedicated clip applier, the metal clip is permanently deformed over the vessel. An example of one such clip is disclosed in U.S. Pat. No. 5,509,920 to Phillips et al. An example of a metallic clip applier is disclosed in U.S. Pat. No. 3,326,216 to Wood in which a forceps-type applier having conformal jaws is used to grip and maintain alignment of the clip during deformation. Such appliers may additionally dispense a plurality of clips for sequential application, as disclosed in U.S. Pat. No. 4,509,518 to McGarry et al.

With the advent of high technology diagnostic techniques using computer tomography (CATSCAN) and magnetic resonance imaging (MRI), metallic clips have been found to interfere with the imaging techniques. To overcome such interference limitations, biocompatible polymers have been increasingly used for surgical clips. Unlike metallic clips, which are usually symmetric, polymeric clips are usually asymmetric in design and hence lack an axis of symmetry. Inasmuch as the plastic clip cannot be permanently deformed for secure closure around a vessel or other tissue, latching mechanisms have been incorporated into the clip design to establish closure conditions and to secure against re-opening of the vessel. For example, well known polymeric clips are disclosed in commonly-owned U.S. Pat. No. 4,834,096 to Oh et al. and U.S. Pat. No. 5,062,846 to Oh et al., the disclosures of which are herein expressly incorporated by reference. These plastic clips generally comprise a pair of curved legs joined at their proximal ends with an integral hinge or heel. The distal ends of the curved legs include interlocking latching members. For example, the distal end of one leg terminates in a lip or hook structure into which the distal end of the other leg securely fits to lock the clip in place.

The distal ends of the clips taught by Oh et al. also include lateral bosses that are engaged by thejaws of the clip applier. A clip applier specifically designed for asymmetric plastic clips is used to close the clip around the tissue to be ligated, and to latch or lock the clip in the closed condition. In operation, the jaws of this clip applier are actuated into compressing contact with the legs of the clip. This causes the legs to pivot inwardly about the hinge, thereby deflecting the hook of the one leg to allow reception therein of the distal end of the other leg. A clip applier designed for use with asymmetric plastic clips in an open (i.e., non-endoscopic) surgical procedure is disclosed in commonly-owned U.S. Pat. No. 5,100,416 to Oh et al.

In addition to compatibility with sophisticated diagnostic techniques, asymmetric clips have other advantages over symmetric clips. For example, because asymmetric clips are formed from polymeric materials, the mouths of asymmetric clips can be opened wider than the mouths of symmetric clips. This allows a surgeon to position the clip about the desired vessel with greater accuracy. In addition, a clip of the type described in U.S. Pat. Nos. 4,834,096 and 5,062,846 can be repositioned before locking the clip on the vessel or before removing the clip from the vessel, in a process referred to as “approximating” the clip.

Although plastic ligating clips are well known in the surgical area and improvements have been made to the ligating clips including providing an interlocking latch member or a sharp protruding distal tip to the hook end in an effort to provide a penetrating plastic ligating clip (see, for example, the aforementioned U.S. Pat. Nos. 4,834,096 and 5,062,846), the latching mechanisms of the prior art plastic ligating clips have a drawback concerning the amount of force that can be applied against them. In certain circumstances, when excess force is applied to the latch mechanism, the latch mechanism may fail. Or, in many cases, especially for larger clips, a surgeon, in attempting to close a clip, with or without a specially fitted applier tool, may not be able to muster the required force to close the clip and properly engage the locking or latching mechanism. Furthermore, empirical evidence has shown that many clips exhibit unreliable or chaotic performance: where, for a given closing force and method of application, some clip successfully close and are applied and other clips either fail to close, prematurely separate, or other fail in their application. Thus, surgeons may benefit from a clip which is more easily and smoothly applied, where the latching or locking mechanism is more easily and reliably engaged to properly apply the clip. The present invention is believed to provide such an improved surgical clip.

### SUMMARY OF THE INVENTION

The foregoing needs are met, to a great extent, by the present invention, wherein in one aspect an apparatus is provided that in some aspects provides a polymeric, surgical clip having first and second curved leg members joined at their proximal end by a hinge portion and movable from an open position to a closed position for clamping a vessel between curved opposing inner surfaces. The clip includes a coating on its exterior which reduces the friction between the leg members and complementary locking mechanisms disposed at the distal end portions of the leg members, and stabilizes the closing of the clip. The coating can be any coating, including a polymer coating such as one which includes polytetrafluoroethylene. The coating may be applied using a solvent such as HFC43-10.

In accordance with one embodiment of the present invention, a surgical ligating clip is provided, including a polymeric clip body having first and second leg members movable from an open position to a closed position. The clip body has an exterior surface including opposing vessel clamping inner surfaces on each leg member. A coating layer is included on at least a portion of the exterior surface of the clip body.

In accordance with another aspect of the present invention, a method of producing a coated surgical ligating clip is provided, including applying a polymer coating to a polymeric clip body having first and second leg members movable from an open position to a closed position. The clip body has an exterior surface including opposing vessel clamping inner surfaces on each leg member.

In accordance with yet another embodiment of the present invention, a surgical ligating clip is provided, having a polymeric clip body having first and second leg members, each leg member having respective proximal and distal end portions. The clip body has an exterior surface and a hinge means coupling the first and second leg members at their respective proximal end portions to enable the first and second leg members to move between an open position of the clip body and a closed position of the clip body. The clip body further includes complementary locking means disposed on the distal end portions of the leg members to maintain the clip body in the closed position when moved to the closed position. The clip body also includes a coating layer on at least a portion of the exterior surface of the clip body.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a perspective view of one embodiment of a surgical clip of the present invention.

FIGS. 2A through 2D are enlarged side elevational views of the clip of FIG. 1.

FIG. 3 shows the clip of FIG. 1 applied to a body vessel.

### DETAILED DESCRIPTION

The invention will now be described with reference to the drawing figures, in which like reference numerals refer to like parts throughout. As used herein, the term “polymeric” shall mean an item that is substantially made of a polymer substance. Furthermore, in accordance with conventional practice, as used herein, the term “proximal” or “proximal end” shall refer to the specified end of a device or its component which is generally closer to the medical personnel handling or manipulating the device as it is intended to be used, and the term “distal” or “distal end” shall refer to the specified end of a device or its component which is opposite the proximal end.

An embodiment in accordance with the present invention provides a polymeric, surgical clip having first and second curved leg members joined at their proximal end by a hinge portion and movable from an open position to a closed position for clamping a vessel between curved opposing inner surfaces. The clip includes a coating on its exterior which reduces the friction between the leg members and complementary locking mechanisms disposed at the distal end portions of the leg members, and stabilizes the closing of the clip. The coating can be any coating, including a polymer coating such as one which includes polytetrafluoroethylene. The coating may be applied using a solvent such as HFC43-10.

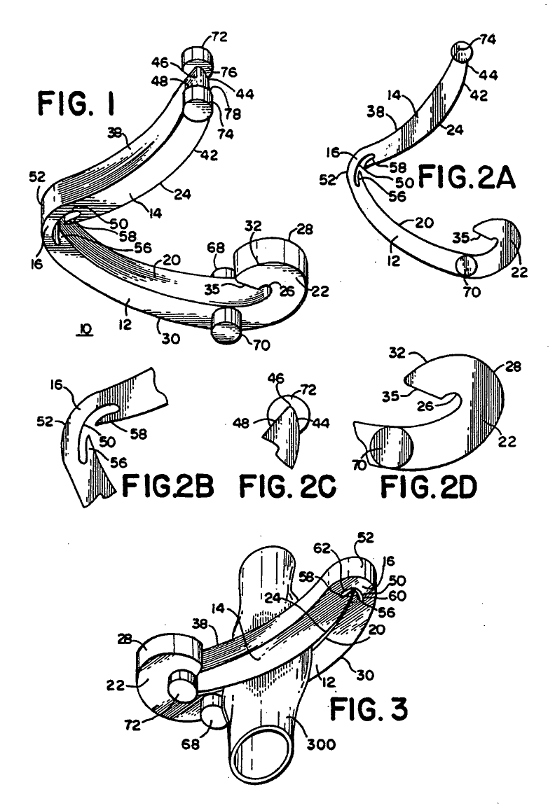

The present invention refers to surgical clips and clips that can be used to ligate or close off body vessels and ducts, as well as other tissues. The clips can have a myriad of designs and configurations. Referring now to FIGS. 1, 2A-2D and 3, a first embodiment of a surgical clip designated generally 10 is shown. It comprises a first curved leg member 12 and a second curved leg member 14 joined at their respective proximal end portions by a hinge portion 16. First leg member 12 has a concave inner surface 20 and a curved hook portion 22 joined at its distal end. The inner surface 24 of second curved leg member 14 is convex and is adapted to be substantially parallel with concave inner surface 20 when the clip is closed, i.e; the radius of curvature of the concave inner surface 20 is substantially the same as inner convex surface 24. The hook portion 22 is curved toward the distal end of second leg member 14.

The inner and outer surfaces 26 and 28, respectively, of the hook portion 22 are continuously curved with the outer surface 28 continuing from the convex outer surface 30 of the first leg member with decreasing radius of curvature while the inner surface 26 continues from the inner concave surface 20 of the first leg member 12 in a decreasing radius of curvature to a constant terminal radius of curvature.

The second leg member 14 has a banana like profile along its length between its inner convex surface 24 and outer concave surface 38. The thickness of the distal end has a rapid reduction in thickness because of the change in radius of curvature of its inner surface 24 at 42 to form a curved inner beveled surface 44 terminating in a tip 46 and the relatively flat beveled surface 48 joining the outer concave surface 38 to the tip 46.

The hinge portion 16 has a substantially reduced thickness between its inner and outer surfaces 50 and 52, respectively, compared with the thicknesses of leg members 12 and 14. The outer surface 52 of the hinge portion 16 connects the outer surfaces 30 and 38 of the leg members 12 and 14, respectively, in a continuous curve. Leg members 12 and 14 can include peninsular portions 56 and 58, respectively, which extend toward the inner surface 50 of the hinge portion 16. The peninsular portions are spaced apart from the proximal ends of the leg members coupled to the hinge portion and they are formed by an extension of the inner surfaces 20 and 24, respectively, of the leg members. The peninsular portions 56 and 58 extend substantially to the inner surface 50 of the hinge portion 16 to form the letter “C” shaped openings 60 and 62 when the clip is closed about the vessel. (See FIG. 3.) Alternatively, the peninsular portions 56 and 58 can be connected at their respective tips to form a continuous channel or strip.

Leg member 12 includes a pair of cylindrical bosses 68 and 70 coupled on opposite sides of leg member 12 intermediate the hook portion 22 and the hinge portion 16, but closer to the hook portion 22 in the present embodiment. The bosses extend laterally away from the leg member.

Leg member 14 includes a pair of cylindrical bosses 72 and 74 located at the tip 46 on opposite sides of the leg member 14 and extending laterally away therefrom. In the preferred embodiment, the coaxial center line of the bosses 72 and 74 passes through the tip portion of the leg member 14. (See FIG. 2C where the boss 74 is removed showing the relationship of the tip 46 to the center of boss 72.) The bosses 72 and 74 extend beyond the tip leaving spaced apart opposing surfaces 76 and 78, respectively. While FIGS. 1 and 2A show the clip 10 in an “open” position, FIG. 3 shows the clip in a “closed” position, as applied to a body vessel, where the complementary locking elements of hook 22 and tip 46 have been actuated to fit together, thereby maintaining the clip 10 in the closed position around body vessel 300.

Additional embodiments of a surgical clip constructed in accordance with the principles of the present invention are shown in commonly owned U.S. Pat. Nos. 4,834,096, 5,062,846, and 6,863,675, as well as commonly-owned published U.S. patent applications having publication Nos. 2005/0165421, 2005/0165422, 2005/0165423, and 2005/0165424, the disclosures of which are herein expressly incorporated by reference in their entirety. The surgical clips can have additional structural features and capabilities, including different complementary locking or latching mechanisms disposed on the distal end portions of legs 12 and 14.

The present invention is directed to a coating that can be applied to at least a portion of the exterior surface of a clip such as clip 10 shown in FIGS. 1-3. By applying a coating to the clip, the friction forces are reduced when the clip is applied though the closing of legs 12 and 14 and the interoperation of hook 22 and tip 46. Lower friction forces enable the clip to be closed more easily, demanding less closing force from the person applying the clip, or the tool being used to apply the clip. The addition of a coating layer on the exterior surface of clips such as clip 10 also lessen the problem of “snow-ploughing” which occurs when the tip 46 is pushed against the exterior surface 32 (see FIG. 2D) of hook portion 22 as the clip is moved to the closed position. As the leg 14 is moved towards leg 12, the tip 46 first contacts the outer surface 32 of hook tip portion 22, deflecting it and compressing it, before slipping past it and sliding past inner surface 35 and into the innermost surface 26 of the hook 22. If too much force is applied to counteract friction forces acting to retard the movement of tip 46 along surfaces 32 and 35, the hook 22 can be deformed or damaged, thereby degrading the quality of the locking mechanism of clip 10, and by consequence the performance of the clip.

To prevent the afore-mentioned problems, the present invention provides a method and apparatus for applying a coating layer to at least a portion of the exterior surface of the clip, so as to improve its properties by reducing surface friction forces, as well as to increase and improve the reliability of the clip as it is moved from the open to the closed position. The coating in question can be any friction-reducing coating, or a coating suited to improve the ability of the clip to be locked and maintain the closed position.

In one embodiment of the present invention, the coating is a polymer coating and can include one or more compounds including polytetrafluoroethylene (PTFE). The coating can be applied using a method whereby a mixture of PTFE and a solvent is applied to the clip and then allowed to dry so as to evaporate the solvent, thereby leaving a coating layer of PTFE on the clip. In one exemplary embodiment, a mixture of Dupont™ DryFilm RA dry lubricant (containing PTFE with hydrofluorocarbon fluid 2,3-Dihydrodecafluoropentane, or “HFC 43-10”) is diluted with Dupont™ Vertrel® XF solvent, itself a pure version of HFC43-10. The DryFilm RA can make up a range of concentrations in the Vertrel® XF solvent, including between 0.5% to 1.5%. The mixture can be applied to the clip body, or any exterior surface portion thereof, using a dip method. The components can then be dried at room temperature, packaged, and sterilized. The HFC43-10 solvent will be evaporated leaving only a surface residual of PTFE as a dry lubricant.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A surgical ligating clip comprising:
a polymeric clip body having first and second leg members movable from an open position to a closed position, the clip body having an exterior surface including opposing vessel clamping inner surfaces on each leg member, and
a coating layer on at least a portion of the exterior surface of the clip body.

2. The clip of claim 1,
wherein the coating layer is comprised of a polymer coating.

3. The clip of claim 2,
wherein the polymer coating is comprised at least in part of polytetrafluoroethylene.

4. The clip of claim 3,
wherein the polymer coating is applied to the clip body by a process including the steps of:
diluting a mixture including one or more polytetrafluoroethylene compounds with a solvent,
applying the mixture to the clip body using a dip method, and
drying the clip body to evaporate the solvent.

5. The clip of claim 4,
wherein the solvent is HFC43-10.

6. The clip of claim 1,
wherein the clip body is made at least in part of a polyacetal polymer.

7. The clip of claim 1,
wherein the clip body is curved, the first and second leg members being curved.

8. The clip of claim 1,
wherein the clip body and leg members each have respective proximal and distal end portions, and
wherein the first and second leg members are joined at their respective proximal end portions by a hinge section of the clip body, and
wherein the distal end portions of the leg members have complementary locking members to maintain the clip in the closed position.

9. The clip of claim 8,
wherein the first leg member terminates at its distal end portion in a deflectable hook member, and the second leg member terminates at its distal end portion in a complimentary locking portion to said hook member whereby when said first and second leg members are moved from the open position to the closed position about the hinge section, the hook member first deflects and then envelops a portion of the distal end of the second leg member to lock the clip in a closed position.

10. A method of producing a coated surgical ligating clip, comprising:
applying a polymer coating to an exterior surface of a polymeric clip body having first and second leg members movable from an open position to a closed position, the exterior surface including opposing vessel clamping inner surfaces on each leg member.

11. The method of claim 10,
wherein the polymer coating is comprised at least in part of polytetrafluoroethylene.

12. The method of claim 11, further comprising:
diluting a mixture of one or more polytetrafluoroethylene compounds with a solvent,
applying the mixture to the clip body using a dip method, and
drying the clip body to evaporate the solvent.

13. The method of claim 12,
wherein the solvent is HFC43-10.

14. The method of claim 10,
wherein the clip body is made at least in part of a polyacetal polymer.

15. The method of claim 10,
wherein the clip body is curved, the first and second leg members being curved.

16. The method of claim 10,
wherein the clip body and leg members each have respective proximal and distal end portions, and
wherein the first and second leg members are joined at their respective proximal end portions by a hinge section of the clip body, and
wherein the distal end portions of the leg members have complementary locking members to maintain the clip in the closed position.

17. The method of claim 16,
wherein the first leg member terminates at its distal end portion in a deflectable hook member, and the second leg member terminates at its distal end portion in a complimentary locking portion to said hook member whereby when said first and second leg members are moved from the open position to the closed position about the hinge section, the hook member deflects about the distal end of said second leg member to lock the clip in a closed position.

18. A surgical ligating clip comprising:
a polymeric clip body having first and second leg members, each having respective proximal and distal end portions, the clip body having an exterior surface,
a hinge means coupling the first and second leg members at their respective proximal end portions to enable the first and second leg members to move between an open position of the clip body and a closed position of the clip body,
complementary locking means disposed on the distal end portions of the leg members to maintain the clip body in the closed position when moved to the closed position, and
a coating layer on at least a portion of the exterior surface of the clip body.

19. The clip of claim 18,
wherein the coating layer is comprised of a polymer coating.

20. The clip of claim 19,
wherein the polymer coating is comprised at least in part of polytetrafluoroethylene.
